(19) United States
(12) Patent Application Publication
(10) Pub. No.: US 2014/0060349 A1
(43) Pub. Date: Mar. 6, 2014

(54) TRANSFER SOLUTION FOR NAIL PRINTING AND NAIL PRINTING METHOD USING THE SAME

(71) Applicant: Hye Sun IM, Seoul (KR)

(72) Inventor: Hye Sun IM, Seoul (KR)

(21) Appl. No.: 14/019,054

(22) Filed: Sep. 5, 2013

(30) Foreign Application Priority Data
Sep. 5, 2012 (KR) 10-2012-0098221

(51) Int. Cl. A61K 8/34; A61Q 3/02; B44C 1/16

(52) U.S. Cl. CPC A61K 8/34; B44C 1/16; A61Q 3/02
USPC 101/34; 424/61

(57) ABSTRACT

Disclosed therein are a transfer solution for nail printing and a nail printing method using the same which can transfer and stick characters, figures, images or photographs printed on printing paper onto nail polish. The transfer solution for nail printing includes: i) an organic solvent for instantaneously separating ink from paper by deteriorating an adhesive force between ink and paper and for transferring the ink onto nail polish while keeping a printed form of the ink; and ii) a viscosity agent for sticking the ink separated from the paper onto the nail polish, thereby providing various nail printing effects because it can transfer various printed matters onto the nail polish while keeping their colors and shapes as they are.

### CROSS REFERENCE TO RELATED APPLICATIONS

This application claims priority under 35 USC 119 and/or 365 of Korean Patent Application No. 10-2012-0098221 filed on Sep. 5, 2012. The contents of the above-identified application are relied upon and incorporated herein by reference in their entirety.

### BACKGROUND OF THE INVENTION

1. Field of the Invention

The present invention relates to a transfer solution for nail printing and a nail printing method using the same which can transfer and stick characters, figures, images or photographs printed on printing paper onto nail polish, and more particularly, to a transfer solution for nail printing, which includes: i) an organic solvent for instantaneously separating ink from paper by deteriorating an adhesive force between ink and paper and for transferring the ink onto nail polish while keeping a printed form of the ink; and ii) a viscosity agent for sticking the ink separated from the paper onto the nail polish, thereby providing various nail printing effects because it can transfer various printed matters onto the nail polish while keeping their colors and shapes as they are.

2. Background Art

In general, consumers use various colors of nail polish according to their life styles and clothes and places so as to coordinate well and provide harmony in general look. Moreover, recently, nail art for expressing beautiful patterns or colors on the surfaces of nails using nail polish of various colors is a growing trend among people who want to break from the dull nail varnish by the existing solid-color nail polish.

However, in the case that the user wants to replace the nail polish previously painted on the nail with nail polish of other color, because the nail polish is firmly stuck on the surface of the nail after dried when the user paints the nail polish on the nail, the user has to paint nail polish of another color after cleaning the previously painted nail polish off with a chemical agent. Therefore, because the process of cleaning the nail polish off is inconvenient, most of the users keep the nail polish of the same color for a long time even though they are not satisfied with the existing color of the nail polish.

In other words, conventionally, the users can paint nail polish of a wanted color after cleaning the previously painted nail polish off with the chemical agent in the case that the user wants to colors or shapes of the nail polish according to the user's styles, clothes or places, and hence, the user feels inconvenience in changing colors or shapes of the nail polish according to the user's needs because the process of changing the colors or shapes of the nail polish is very inconvenient and the user has to spend a lot of time. Accordingly, most of the users frequently feel difficulty in coordinating colors or shapes of nail polish with colors of their clothes or the users' accessories.

### CITED REFERENCES

Cited reference 1: Korean Patent Laid-open No. 2003-0062494
Cited reference 2: Korean Patent Laid-open No. 2012-0006765

### SUMMARY OF THE INVENTION

Considering that nail art for expressing beautiful patterns or colors on the surfaces of nails using nail polish of various colors is a recent trend among people who want to break from the dull nail varnish by the existing solid-color nail polish, the users need nail polish coordinated with the users' styles, such as colors of clothes or accessories, and hence, there is a need to easily change colors or shapes of nail polish according to a change of styles. Therefore, in consideration of such needs, the inventor of the present invention invented a transfer solution for nail printing which can provide various nail printing effects by transferring various printed matters onto nail polish while keeping their colors and shapes as they are even after the user previously painted nail polish on the nails.

The transfer solution manufactured by mixing ingredients at a proper content ratio instantaneously separates ink from paper by deteriorating an adhesive force between ink and paper of a printed matter and, at the same time, transfers and sticks the ink onto nail polish while keeping colors and shapes of the ink as they are, thereby enhancing an esthetic effect of the nails by changing colors and shapes of the nails easily according to needs because the transfer solution can provide various changes on the nail polish at any time using various printed matters even after the user previously painted nail polish on the nails.

To achieve the above objects, the present invention provides a transfer solution for nail printing, which can transfer and stick characters, figures, images or photographs printed on printing paper onto nail polish, including: i) an organic solvent for instantaneously separating ink from paper by deteriorating an adhesive force between ink and paper and for transferring the ink onto nail polish while keeping printed colors and shapes of the ink as they are; and ii) a viscosity agent for sticking the ink separated from the paper onto the nail polish.

In this instance, the organic solvent may be alcohol, and it is preferable that the alcohol content is in the range of 45% to 85% by weight of the gross weight. It is preferable that the content of the viscosity agent is in the range of 0.05% to 1% by weight of the gross weight. Moreover, the transfer solution may further include a solubilizer for aiding the viscous activity of the viscosity agent, and it is preferable that the content of the solubilizer is in the range of 0.05% to 1% by weight of the gross weight.

Furthermore, the transfer solution may further include a surfactant for stabilizing mixing of the organic solvent and the viscosity agent, and it is preferable that the content of the surfactant is in the range of 0.1% to 5% by weight of the gross weight.

Additionally, the transfer solution may further include a nourishing agent, a moisturizer, a preservative, and an antioxidant. In a preferred embodiment, the transfer solution consists of 45% to 85% by weight of ethanol, 10% to 40% by weight of purified water, 0.05% to 1% by weight of carbomer, 0.05% to 1% by weight of triethanolamine, 0.1% to 5% by weight of polysorbate, 0.1% to 5% by weight of keratin, 0.1% to 5% by weight of butylene glycol, 0.1% to 1% by weight of phenoxyethanol, and 0.1% to 1% by weight of sodium EDTA.

In another aspect of the present invention, the present invention provides a nail printing method including the steps of: i) manicuring to paint the background color on the nails; ii) completely drying the nail polish painted on the nails; iii) painting the transfer solution of the present invention on printing paper or the surfaces of the nails; iv) positioning the printing paper, on which the transfer solution is painted, on the nails in such a way that a printed ink part gets in contact with the nail polish on the nails; v) pressurizing the printing paper from the top so that the ink part of the printing paper is transferred and stuck onto the nail polish of the nails; and vi) removing the printing paper from the nails.

In this instance, preferably, after the step iv) of positioning the printing paper on the nails, before the v) step of pressurizing the printing paper, in order to make the ink part of the printing paper be sufficiently separated from the paper and transferred and stuck onto the nail polish, the nail printing method further comprises a step of painting the transfer solution on the rear surface of the printing paper once more.

Moreover, in the step vi), in order to completely remove the residue of the paper remaining on the nails after removing the printing paper from the nails, the nail printing method further comprises a step of painting the transfer solution on the nails and rubbing the residue of the paper to take off the residue of the paper. Additionally, in the step v), the printing paper is pressurized from the top for more than 10 seconds so as to sufficiently transfer and stick the ink part of the printing paper onto the nail polish.

In addition, the printing paper has characters, figures, images or photographs printed on paper in ink.

As described above, the transfer solution for nail printing according to the present invention can instantaneously separate ink from paper by deteriorating an adhesive force between ink and paper of printing paper and transfer and stick printed matters of ink onto nail polish while keeping printed colors and shapes of the ink. Therefore, the transfer solution according to the present invention can enhance an esthetic effect of the nails by changing colors and shapes of the nails easily according to needs because the transfer solution can provide various changes on the nail polish using various printed matters, such as newspaper or the user's own printed matters, even after the user previously painted nail polish on the nails.

Particularly, because the transfer solution can transfer the printed matters onto the nail polish as they are without needing for the user to directly draw shapes on the nails, there is no limitation in expression and colors and the user can easily express various shapes on the nail polish without the aids of others.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIGS. 1 to 5 are photographs showing a process of nail printing according to the present invention; and

FIGS. 6a to 6c are photographs showing nail printing according to preferred embodiments of the present invention.

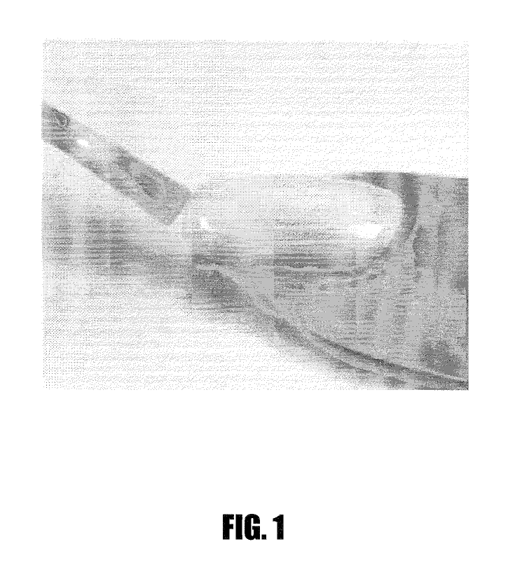

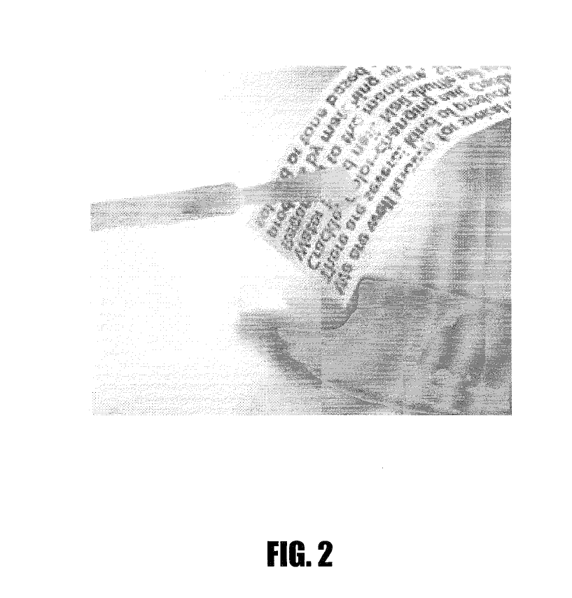

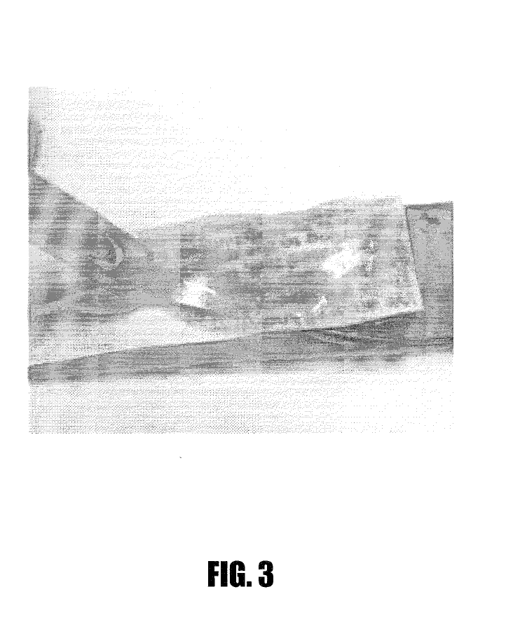

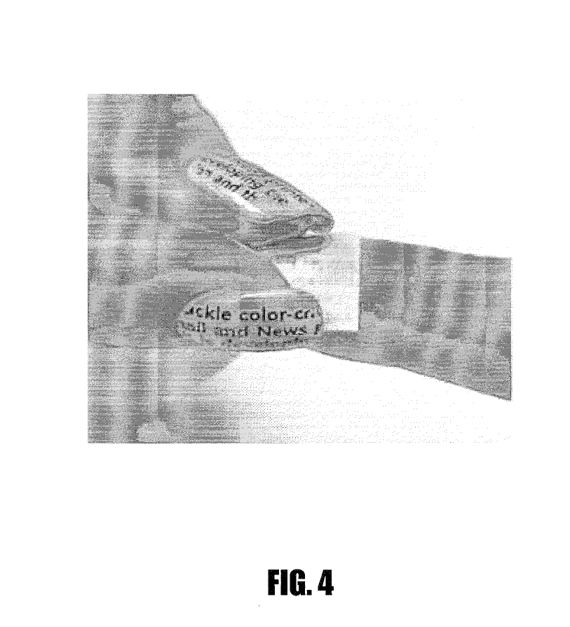

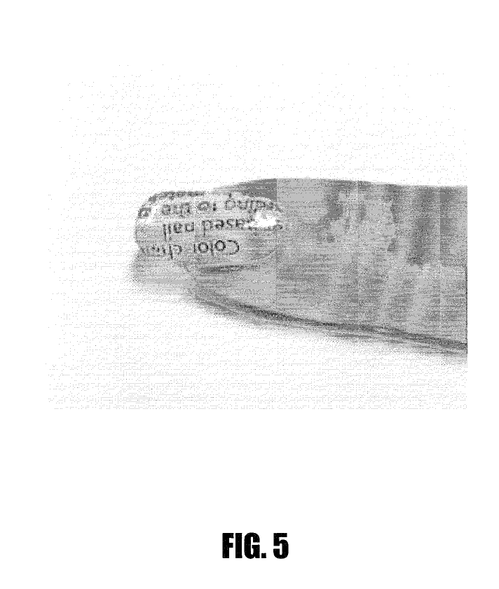

### DETAILED DESCRIPTION OF THE PREFERRED EMBODIMENT

Reference will be now made in detail to the preferred embodiment of the present invention with reference to the attached drawings. It will be understood that words or terms used in the specification and claims shall not be interpreted as the meaning defined in commonly used dictionaries but should be interpreted as having a meaning that is consistent with their meaning in the context of the relevant art and the technical idea of the present invention.

The transfer solution for nail printing is to transfer and stick characters, figures, images or photographs printed on printing paper onto nail polish, and the transfer solution for nail printing includes: i) an organic solvent for instantaneously separating ink from paper by deteriorating an adhesive force between ink and paper and for transferring the ink onto nail polish while keeping printed colors and shapes of the ink as they are; and ii) a viscosity agent for sticking the ink separated from the paper onto the nail polish.

In this instance, the organic solvent may be alcohol, and it is preferable that the alcohol content is in the range of 45% to 85% by weight of the gross weight. If the alcohol content is less than 45% to 85% by weight, ink does not sufficiently separated from paper, and if the alcohol content is more than 45% to 85% by weight, it is difficult to obtain a satisfied nail printing effect due to deterioration in adhesion with nail polish.

Furthermore, the viscosity agent may be various viscous constituents industrially usable, such as carbomer, and it is preferable that the content of the viscosity agent is in the range of 0.05% to 1% by weight of the gross weight. Additionally, in order to aid the viscous activity of the viscosity agent, various solubilizers industrially usable, such as triethanolamine, may be additionally used. It is preferable that the content of the solubilizer is in the range of 0.05% to 1% by weight of the gross weight. If the contents of the viscosity agent and the solubilizer are less than the above, adhesive force between the ink and the nail polish is deteriorated, but if the contents of the viscosity agent and the solubilizer are more than the above, the ink is not sufficiently separated from paper and is crumbled without keeping its shape.

In the meantime, in order to stabilize mixing of the organic solvent and the viscosity agent so that the ingredients can sufficiently show their functions, a surfactant may be added, and in this instance, the surfactant may be various surface-active constituents industrially usable, such as polysorbate. In this instance, it is preferable that the content of the surfactant is in the range of 0.1% to 5% by weight of the gross weight. If the content of the surfactant is more or less than the above, mixing of the organic solvent and the viscosity agent is not stabilized, and hence, the ingredients cannot sufficiently show their functions.

Besides above, the transfer solution for nail printing according to the present invention may additionally include ingredients used in nail products, such as nourishing agents, moisturizers, preservatives, antioxidants, perfumes, and pigments. Preferably, the transfer solution for nail printing according to the present invention may consist of 45% to 85% by weight of ethanol, 10% to 40% by weight of purified water, 0.05% to 1% by weight of carbomer, 0.05% to 1% by weight of triethanolamine, 0.1% to 5% by weight of polysorbate, 0.1% to 5% by weight of keratin, 0.1% to 5% by weight of butylene glycol, 0.1% to 1% by weight of phenoxyethanol, and 0.1% to 1% by weight of sodium EDTA.

In the meantime, a nail printing method using the transfer solution according to the present invention includes the steps of: i) manicuring to paint the background color on the nails; ii) completely drying the nail polish painted on the nails; iii) painting the transfer solution of the present invention on printing paper or the surfaces of the nails; iv) positioning the printing paper, on which the transfer solution is painted, on the nails in such a way that a printed ink part gets in contact with the nail polish on the nails; v) pressurizing the printing paper from the top so that the ink part of the printing paper is transferred and stuck onto the nail polish of the nails; and vi) removing the printing paper from the nails.

In this instance, after the step iv) of positioning the printing paper on the nails, before the v) step of pressurizing the printing paper, in order to make the ink part of the printing paper be sufficiently separated from the paper and transferred and stuck onto the nail polish, it is preferable to add a step of painting the transfer solution on the rear surface of the printing paper once more.

Moreover, in the step vi), in order to completely remove the residue of the paper remaining on the nails after removing the printing paper from the nails, a step of painting the transfer solution on the nails and rubbing the residue of the paper to take off the residue may be added. The transfer solution can completely remove the residue of paper because it does not melt the ink and nail polish constituents but is absorbed and expanded into the paper.

Furthermore, in the step v), it is preferable to pressurize the printing paper from the top for more than 10 seconds so as to sufficiently transfer and stick the ink part of the printing paper onto the nail polish. In this instance, because the transfer solution does not melt the ink and nail polish constituents but is absorbed and expanded into the paper, the printing paper does not slip or move on the nails. Therefore, even though a user directly does the nail painting without any aid, the user can obtain a wanted shape because the ink part is not moved or crumbled.

In this instance, the printing paper may be all kinds of printed matters that characters, figures, images or photographs are printed on paper in ink, such as infosheets like newspaper or the user's own printed matters. Accordingly, the transfer solution according to the present invention can provide various changes to the nail polish using wanted printing paper so as to enhance the aesthetic effect of the nails by easily changing colors and shapes of the nails according to needs.

Particularly, because the transfer solution can transfer the printed matters onto the nail polish as they are without needing for the user to directly draw shapes on the nails, there is no limitation in accurate expression of lines or colors and the user can easily express a wanted shape without any aid.

Hereinafter, through the preferred embodiments of the present invention, effects of the present invention will be described in detail. However, it would be understood that the embodiments of the present invention have been described exemplarily and the technical scope of the present invention is not limited to the illustrated embodiments.

### Embodiment 1

### Manufacturing of Transfer Solution

The transfer solution for nail printing according to the present invention was manufactured by mixing 65.000% by weight of ethanol, 30.930% by weight of purified water, 0.150% by weight of carbomer, 0.150% by weight of triethanolamine, 0.500% by weight of polysorbate 80, 1.000% by weight of keratin, 1.500% by weight of butylene glycol, 0.150% by weight of phenoxyethanol, 0.50% by weight of sodium EDTA, 0.500% by weight of perfume, and 0.070% by weight of Red 227.

### Embodiment 2

### Nail Printing Using Transfer Solution

Nail polish for background color was painted on the nails and completely dried. After that, the transfer solution manufactured in the first embodiment was painted on the printing paper and the surfaces of the nails, and the printing paper painted with the transfer solution was positioned on the nail polish in such a way that the ink part printed on the printing paper got in contact with the nail polish.

The transfer solution was painted on the rear surface of the printing paper once more, and then, the printing paper was pressurized from the top for about 20 seconds, such that the ink part of the printing paper was transferred and stuck onto the nail polish on the nails. The printing paper was removed from the nails, and then, in order to completely remove the residue of the paper remaining on the nails, the user painted the transfer solution on the nails, and then, rubbed the residue of the paper so as to completely take off the residue of paper.

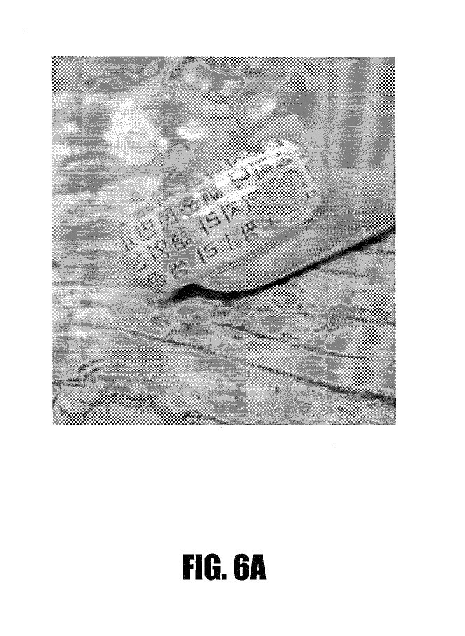

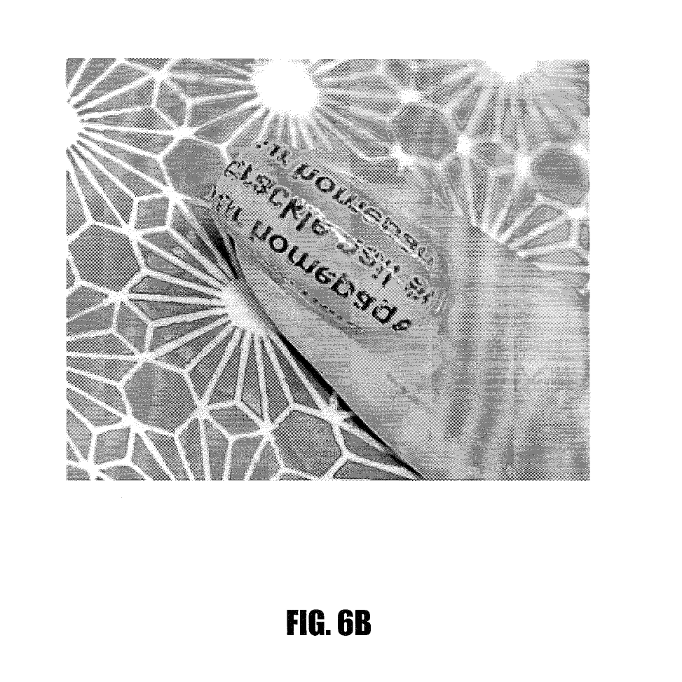

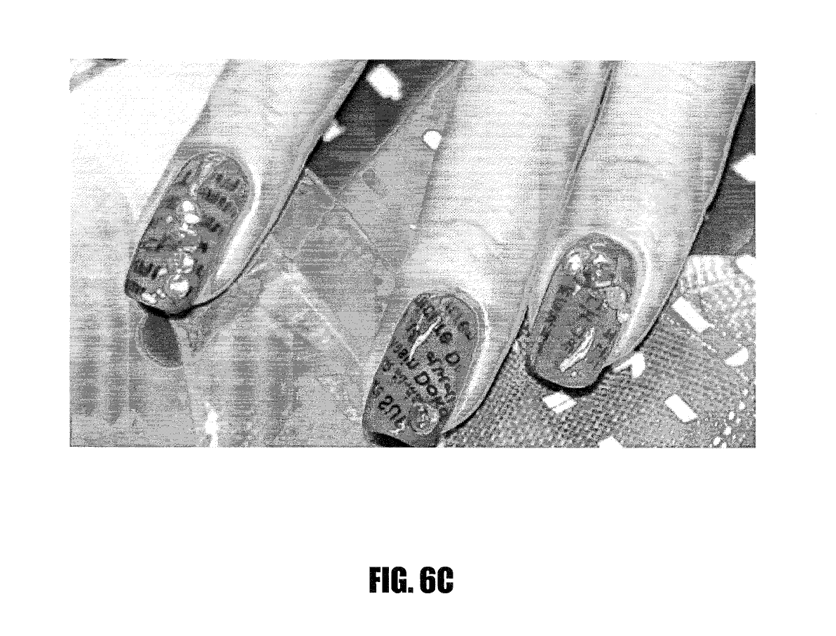

FIGS. 6a to 6c are photographs of nail printing according to the above embodiment, showing that the printed contents were transferred onto the nail polish clearly.

As described above, while the present invention has been particularly shown and described with reference to the specific embodiments thereof, it will be understood by those of ordinary skill in the art that various changes, modifications and equivalents may be made therein without departing from the technical idea and scope of the present invention as defined by the following claims and all of the changes belong to the technical scope of the present invention.

What is claimed is:

1. A transfer solution for nail printing, which transfers and sticks characters, figures, images or photographs printed on printing paper onto nail polish, the transfer solution for nail printing comprising:
i) an organic solvent for instantaneously separating ink from paper by deteriorating an adhesive force between ink and paper and for transferring the ink onto nail polish while keeping printed colors and shapes of the ink as they are; and
ii) a viscosity agent for sticking the ink separated from the paper onto the nail polish.

2. The transfer solution according to claim 1, wherein the organic solvent is alcohol.

3. The transfer solution according to claim 2, wherein the alcohol content is in the range of 45% to 85% by weight of the gross weight.

4. The transfer solution according to claim 1, wherein the content of the viscosity agent is in the range of 0.05% to 1% by weight of the gross weight.

5. The transfer solution according to claim 1, further comprising:
a solubilizer for aiding the viscous activity of the viscosity agent.

6. The transfer solution according to claim 5, wherein the content of the solubilizer is in the range of 0.05% to 1% by weight of the gross weight.

7. The transfer solution according to claim 1, further comprising:
a surfactant for stabilizing mixing of the organic solvent and the viscosity agent.

8. The transfer solution according to claim 7, wherein the content of the surfactant is in the range of 0.1% to 5% by weight of the gross weight.

9. The transfer solution according to claim 1, further comprising:
a nourishing agent, a moisturizer, a preservative, and an antioxidant.

10. The transfer solution according to claim 9, wherein the transfer solution consists of 45% to 85% by weight of ethanol, 10% to 40% by weight of purified water, 0.05% to 1% by weight of carbomer, 0.05% to 1% by weight of triethanolamine, 0.1% to 5% by weight of polysorbate, 0.1% to 5% by weight of keratin, 0.1% to 5% by weight of butylene glycol, 0.1% to 1% by weight of phenoxyethanol, and 0.1% to 1% by weight of sodium EDTA.

11. A nail printing method comprising the steps of:
i) manicuring to paint the background color on the nails;
ii) completely drying the nail polish painted on the nails;
iii) painting the transfer solution of the present invention on printing paper or the surfaces of the nails;
iv) positioning the printing paper, on which the transfer solution is painted, on the nails in such a way that a printed ink part gets in contact with the nail polish on the nails;
v) pressurizing the printing paper from the top so that the ink part of the printing paper is transferred and stuck onto the nail polish of the nails; and
vi) removing the printing paper from the nails.

12. The nail printing method according to claim 11, wherein after the step iv) of positioning the printing paper on the nails, before the v) step of pressurizing the printing paper, in order to make the ink part of the printing paper be sufficiently separated from the paper and transferred and stuck onto the nail polish, the nail printing method further comprises a step of painting the transfer solution on the rear surface of the printing paper once more.

13. The nail printing method according to claim 11, wherein in the step vi), in order to completely remove the residue of the paper remaining on the nails after removing the printing paper from the nails, the nail printing method further comprises a step of painting the transfer solution on the nails and rubbing the residue of the paper to take off the residue of the paper.

14. The nail printing method according to claim 11, wherein in the step v), the printing paper is pressurized from the top for more than 10 seconds so as to sufficiently transfer and stick the ink part of the printing paper onto the nail polish.

15. The nail printing method according to claim 11, wherein the printing paper has characters, figures, images or photographs printed on paper in ink.
